(19) United States
(12) Patent Application Publication
(10) Pub. No.: US 2010/0249820 A1
(43) Pub. Date: Sep. 30, 2010

(54) LANCING DEVICES

(75) Inventor: Timothy Simon Evans, Oxfordshire (GB)

Correspondence Address: YOUNG & THOMPSON, 209 Madison Street, Suite 500, Alexandria, VA, 22314, US

(73) Assignee: OWEN MUMFORD LIMITED, Oxford (GB)

(21) Appl. No.: 12/666,218

(22) PCT Filed: Jun. 20, 2008
(86) PCT No.: PCT/GB2008/002126

§ 371 Date: May 10, 2010

(30) Foreign Application Priority Data
Jun. 22, 2007 (GB) 0712107.2

(51) Int. Cl. A61B 17/32

(52) U.S. Cl. 606/182

(57) ABSTRACT

A lancet holder 30 for use in a lancing device releasably to hold a lancet 32 includes a generally cylindrical sleeve 44 from the inner surface of which project three equi-spaced guide ribs/deflectors 50. Upon introducing a lancet 32 into the lancet holder the guide ribs/deflectors 50 nudge the lancet angularly to ensure that the ribs 36 on the lancet are slidably received in the gaps between the guide ribs/deflectors.

This invention relates to lancing devices and in particular, but not exclusively, to such devices designed to receive a disposable or replaceable lancet for skin pricking.

In applications where a user needs to prick the skin regularly to draw a bead of blood for test purposes (e.g. blood glucose level), it is common to provide a lancing device comprising a housing containing a lancet drive mechanism and a lancet holder which receives in use a disposable lancet. The user loads a disposable lancet into the device, cocks it and then triggers the device to cause the lancet to prick the skin. After use, the lancet is disposed of to prevent contamination or infection. It is also known to provide the lancet with a protective cap which is moulded over the lancet needle, so that the lancet can be handled safely prior to loading it in the lancing device and to keep it sterile. Once loaded, the cap can be twisted off to expose the needle. After the device has been fired, the cap can be reattached to the lancet to cover the needle prior to disposal.

It is therefore important to provide some means of preventing the lancet rotating in the holder when the cap is twisted off. Also, for quality control and product liability purposes a lancing device should only used with disposable lancets specifically designed for use therewith. With the small dimensions of the lancet and the lancet holder it can be difficult for non-technical patients or those with limited vision or impaired dexterity to correctly align with the lancet holder a lancet having a non-circular shaft. Likewise, it is important to ensure that the lancet does not jam in the lancet holder as this could encourage the user to use excessive force, possibly exposing the lancet needle prematurely or bending the tip whilst still capped, or in the lancet being too far forward in the lancet holder thereby leading to malfunction.

Accordingly, in one aspect, this invention provides a lancing device comprising a housing containing a lancet drive mechanism and a lancet holder for receiving in use a disposable lancet, said lancet having a non-circular shaft, said lancet holder having a wall region defining an elongate space for receiving the shaft of said lancet, the lancet holder having at least one internally projecting rib for resisting rotation of a lancet when received in said holder, and the internally projecting rib having a forward deflecting region to urge said lancet shaft angularly to orient said lancet such that said lancet in use is aligned angularly with said elongate space during insertion.

In one arrangement said lancet has a non-circular shaft comprising two or more radial flanges extending longitudinally. to define a plurality of intervening elongate relieved flute regions. In another arrangement said lancet may have a shaft of rectangular cross-section, for example of the type shown in US Re35803 or U.S. Pat. No. 5,318,584.

Preferably said radial flanges are equiangularly spaced, and said projecting rib or ribs of said lancet holder are correspondingly spaced.

The ribs may taper in width and radial dimension in a forward region thereof to define a pointed deflecting region.

The lancet holder preferably includes resilient detent means for releasably retaining a lancet in use, for example two diametrically opposed resilient detent means. The resilient detent means conveniently comprise a flexible tongue portion formed in the wall region of the holder.

Preferably the holder has an odd number of ribs, and one of said resilient detent means is aligned with, or forms part of, one of said ribs, and the diametrically opposed resilient detent is disposed in a gap between two of said ribs.

Whilst the invention has been described above, it extends to any inventive combination or sub-combination of the features set out above or in the following description.

The invention may be performed in various ways, and an embodiment thereof will now be described by way of example only, reference being made to the accompanying drawings in which:—

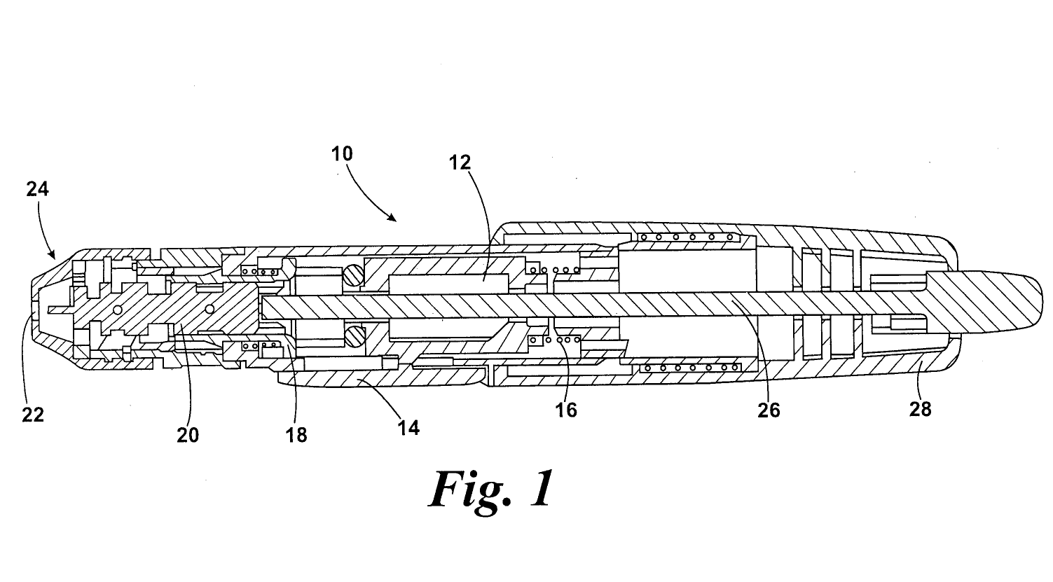

FIG. 1 is a cross sectional view of a prior art lancing device disclosed in published International patent application No. WO01/13794;

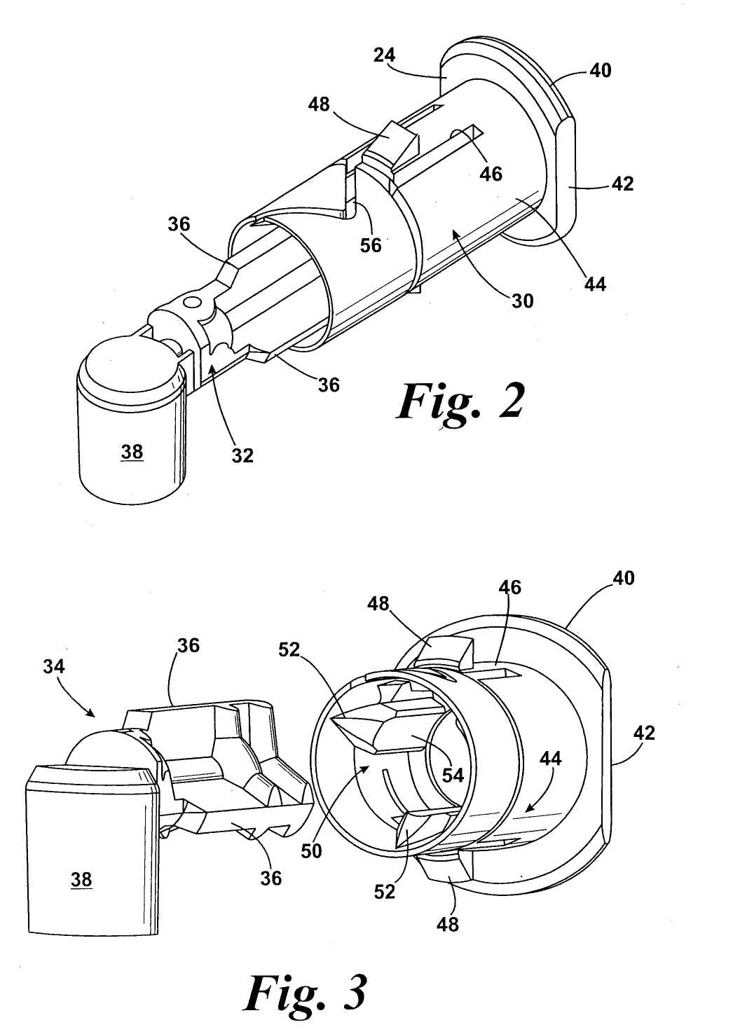

FIG. 2 is a perspective view on a lancet holder of a lancing device of an embodiment of this invention, with a lancet loaded;

FIG. 3 is a perspective view similar to FIG. 2 but showing the lancet prior to insertion into the lancet holder;

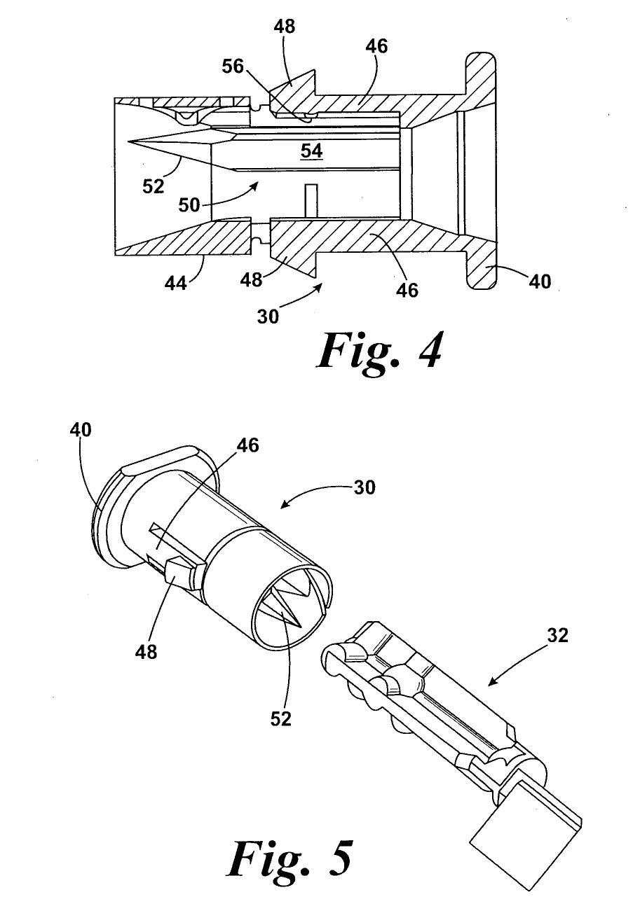

FIG. 4 is a detailed cross sectional view on the lancet holder of FIGS. 2 and 3, and

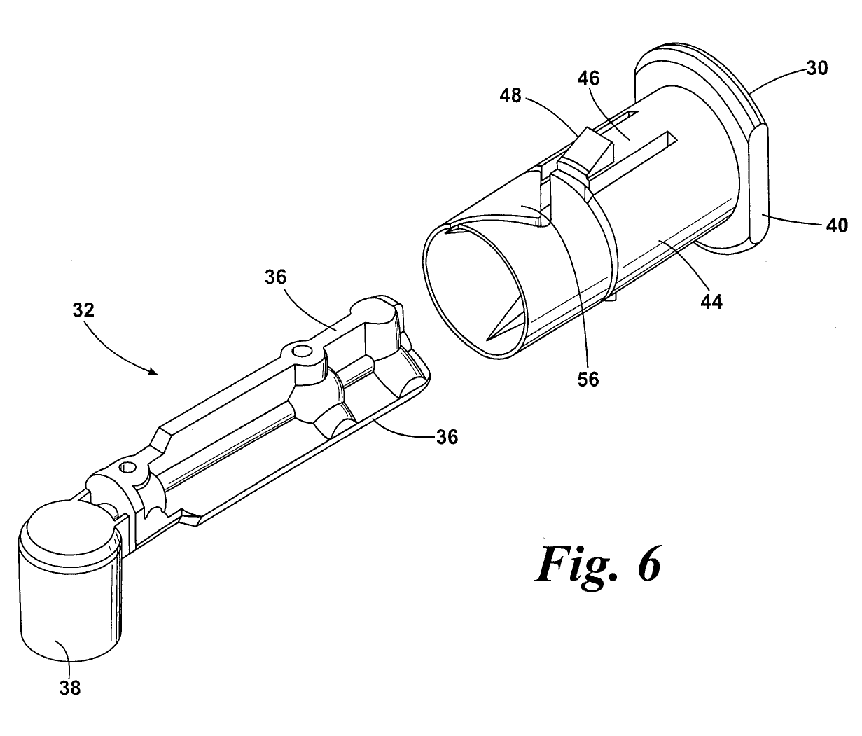

FIGS. 5 and 6 are perspective views of the lancet and lancet holder of FIGS. 2 to 4, from different aspects.

Referring initially to FIG. 1 there is shown a lancing device of the type described, in WO01/13794. This device comprises a barrel 10 in which a hammer 12 released by a trigger mechanism 14 can be shot forwards by a compressed spring 16 to impact on the rear end of a lancet holder 18 to drive it forward so a lancet 20 held therein can momentarily project its needle tip through an aperture 22 in a nose piece 24. After firing, the nose piece and the attached collar may be removed and the lancet 20 pushed forwardly out of the lancet holder 18 by an ejector rod 26. A new lancet 20 can be inserted following removal of the nose piece 24 and pushed into the lancet holder 18. In this particular arrangement, the hammer is re-cocked by pulling back a sleeve 28 on the housing which pulls the hammer 12 back to its cocked position as set out in more detail in WO01/13794.

In the illustrated embodiment of this invention, the lancet holder 18 and lancet 20 are replaced by the lancet holder 30 and lancet 32 as shown in FIGS. 2 to 6. Otherwise the lancing device operates substantially as previously described, although of course the invention may be used in any of a wide range of different lancing devices where a lancet is inserted into a holder.

Referring now to FIGS. 2 to 6, the lancet comprises an elongate shaft 34 comprising three radial flanges 36 equiangularly spaced to define flute regions between them. The lancet has a cap 38 integrally moulded over the tip, the cap being removable by twisting. The lancet holder has a rear outwardly turned rim 40 for receiving the impact of the hammer, the rim 40 having flats 42. to prevent it rotating within the housing. Forwardly of the rim 40 there is a generally cylindrical sleeve 44 having two diametrically opposed tongues 46 capable of radial resilient movement and having on their forward ends barbs 48. The barbs 48 cooperate with a forwardly facing shoulder on the front of the housing 10 of the lancing device to limit rearward movement of the lancet body. Internally of the cylindrical sleeve 44 there are provided three equi-spaced guide ribs/deflectors 50. Each guide rib/deflector 50 comprises a pointed alignment region 52 forwardly of a key rib 54. A V 56 is cut in the forward end of the sleeve and communicates with one of the slots defining the upper tongue as seen in FIG. 6.

The guide ribs/deflectors 50 are disposed round the sleeve such that one of them is angularly aligned with one of the resilient teeth 46, with the other resilient tooth 46 lying midway between two guide ribs/deflectors 50. The tooth 46 lying between the two guide ribs/deflectors is provided with an inwardly directed pip 56, (see FIG. 4).

In use, when a lancet 32 is offered up to the lancet holder, in any configuration, if the ribs 36 are not aligned with the gaps between the guide ribs/deflectors 50 on the holder 50, the pointed alignment regions 52 will nudge the lancet angularly to effect such alignment. As the lancet is pushed home into the lancet holder, the pip 56 on the upper tooth (as seen in FIG. 4) rides on one of the ribs 36 to provide a frictional hold.

When the lancet is fully home, the cap may be twisted off and rotation of the lancet relative to the holder is prevented by inter-engagement of the radial flanges 36 with the guide ribs/deflectors 50 and by the flats 42 on the lancet holder engaging corresponding surfaces on the interior of the housing of the lancing device.

1. A lancing device comprising a housing containing a lancet drive mechanism and a lancet holder for receiving in use a disposable lancet, said lancet having a non-circular shaft, said lancet holder having a wall region defining an elongate space for receiving the shaft of said lancet, the lancet holder having at least one internally projecting rib for resisting rotation of a lancet when received in said holder, and the internally projecting rib having a forward deflecting region to urge said lancet shaft angularly to orient said lancet such that said lancet in use is aligned angularly with said elongate space during insertion.

2. A lancing device according to claim 1, wherein said lancet holder comprises two or more radial flanges extending longitudinally to define a plurality of intervening elongate relieved flute regions.

3. A lancet device according to claim 2, wherein said radial flanges are equiangularly spaced, and said projecting rib or ribs of said lancet holder are correspondingly spaced.

4. A lancing device according to claim 2, wherein said ribs taper in width and radial dimension in a forward region thereof to define a pointed deflecting region.

5. A lancing device according to claim 1, wherein said lancet holder includes resilient detent means for releasably retaining a lancet in use.

6. A lancing device according to claim 5 wherein said lancet holder comprises two diametrically opposed resilient detent means.

7. A lancing device according to claim 5, wherein said resilient detent means comprise a flexible tongue portion formed in the wall region of the holder.

8. A lancing device according to claim 6, wherein the holder has an odd number of ribs and wherein one of said resilient detent means is aligned with, or forms part of, one of said ribs, and the diametrically opposed resilient detent is disposed in a gap between two of said ribs.

9. A lancing device according to claim 3, wherein said ribs taper in width and radial dimension in a forward region thereof to define a pointed deflecting region.

10. A lancing device according to claim 6, wherein said resilient detent means comprise a flexible tongue portion formed in the wall region of the holder.

11. A lancing device according to claim 7, wherein the holder has an odd number of ribs and wherein one of said resilient detent means is aligned with, or forms part of, one of said ribs, and the diametrically opposed resilient detent is disposed in a gap between two of said ribs.
